(19) United States
(12) Patent Application Publication
(10) Pub. No.: US 2021/0321998 A1
(43) Pub. Date: Oct. 21, 2021

(54) SURGICAL RETRACTOR TOOL FOR ENDOSCOPIC AND MINIMALLY-INVASIVE SURGERIES

(71) Applicants: Ronney ABAZA, Dublin, OH (US); Matthew C FERRONI, Dublin, OH (US); Jamison FLOAT, Lewis Center, OH (US); Donald MEVES, Gahanna, OH (US)

(72) Inventors: Ronney ABAZA, Dublin, OH (US); Matthew C FERRONI, Dublin, OH (US); Jamison FLOAT, Lewis Center, OH (US); Donald MEVES, Gahanna, OH (US)

(21) Appl. No.: 17/232,864

(22) Filed: Apr. 16, 2021

Related U.S. Application Data
(60) Provisional application No. 63/010,872, filed on Apr. 16, 2020.

(51) Int. Cl. A61B 17/02

(52) U.S. Cl. CPC A61B 17/0218; A61B 2017/0287

(57) ABSTRACT

A surgical retractor tool includes an atraumatic device, and anchor, and a connector coupled at a first end to the atraumatic device and coupled at a second, opposite end to the anchor. The atraumatic device is transformable between a transition configuration, in which the atraumatic device is characterized by a substantially linear shape, and a deployed configuration, in which the atraumatic device is characterized by a non-linear shape. The non-linear shape is configured to releasably engage a first anatomical portion of a patient. The anchor is configured to be releasably secured to a second anatomical portion of the patient. In the transition configuration, the surgical retractor tool is characterized by a substantially linear shape defined along a longitudinal axis extending from an end of the anchor to an end of the atraumatic device.

### CROSS-REFERENCE TO RELATED APPLICATIONS

This application is related to, and claims the benefit of priority from, U.S. Provisional Application Ser. No. 63/010872, filed 16 Apr. 2020, the disclosure of which is incorporated herein by reference in its entirety.

### FIELD OF THE INVENTION

The present invention pertains to the field of surgical retractor tools, and more particularly to surgical retractor tools used for endoscopic and minimally-invasive surgeries, such as, for example and without limitation, laparoscopic and robotic surgeries.

### BACKGROUND

Surgical procedures are used to treat and cure a wide range of diseases, conditions, and injuries. Surgery often requires access to internal tissue through open surgical procedures or endoscopic surgical procedures. The term “endoscopic” comprehends to all types of minimally invasive surgical procedures including laparoscopic, thoracoscopic, arthroscopic, natural orifice intraluminal, and natural orifice transluminal procedures.

Surgical access devices are often used to facilitate surgical manipulation of internal tissue. For example, trocars are often used to provide a port through which endoscopic surgical instruments are passed, while retractors are used to maintain or displace the location of tissue or other anatomical portion of the patient during surgery in endoscopic and minimally invasive surgeries.

With single-port robotic surgery, retraction is limited due to the shorter length of instruments and tethering of all four robotic arms to each other, given that they all enter through a single channel into the body. Moreover, the vector of retraction is limited since the direction of the robotic instruments entering the body is the same.

While retractor tools of numerous types have long been known, a retractor tool for surgeries of the type described herein has heretofore been unknown.

### SUMMARY OF THE DISCLOSURE

The present invention is a surgical retractor tool, comprising an atraumatic device, an anchor, and a connector coupled at a first end to the atraumatic device and coupled at a second, opposite end to the anchor. The atraumatic device is transformable between a transition configuration, in which the atraumatic device is characterized by a substantially linear shape defined along a longitudinal axis of the atraumatic device, and a deployed configuration, in which the atraumatic device is characterized by a non-linear shape defined along the longitudinal axis of the atraumatic device. The non-linear shape of the atraumatic device is configured to releasably engage a first anatomical portion of a patient. The anchor is configured to be releasably secured to a second anatomical portion of the patient. In the transition configuration of the atraumatic device, the surgical retractor tool is characterized by a substantially linear shape defined along a longitudinal axis of the surgical retractor tool extending from an end of the anchor to an end of the atraumatic device.

Per one feature, the connector comprises a length that is manually adjustable relative to the atraumatic device to adjust an overall length of the surgical retractor tool.

Per another feature, the atraumatic device is hingedly coupled to the connector.

According to yet another feature, the connector further comprises an elastic portion that is temporarily deformable in at least the longitudinal axis of the surgical retractor tool. In one form, the elastic portion includes a strain indicator configured to identify temporary deformation of the elastic portion. In one embodiment, the atraumatic device is coupled to the elastic portion.

Per one feature, in the deployed configuration the non-linear shape of the atraumatic device defines a hook configured to releasably engage the first anatomical portion of the patient.

According to another feature, the atraumatic device comprises at least one hinge joint at which the atraumatic device is transformable between the transition configuration and the deployed configuration.

In one form, the at least one hinge joint is provided between first and second sections of the atraumatic device. In one embodiment, the at least one hinge joint comprises a releasable locking joint configured to selectively lock the at least one hinge joint in each of the transition and deployed configurations of the atraumatic device.

According to a further feature, the atraumatic device is characterized by smooth surfaces for releasably engaging the first anatomical portion of the patient.

The anchor is configured to be traumatically secured to the second anatomical portion of the patient. In one form, the anchor is configured as a hook. In another form, the anchor is a clamp that is manually actuatable between a closed, clamped position and an open, unclamped position.

According to another embodiment, the atraumatic device comprises a monolithic length of material which is manually deformable to transform the atraumatic device between the transition configuration and the deployed configuration.

Per one feature, the monolithic length of material is overcoated with an elastomeric material, such as, by way of non-limiting example, silicone.

According to another feature, the surgical retractor tool is, in the transition configuration of the atraumatic device, dimensioned to be received into a patient through a surgical port. Per one embodiment, the surgical retractor tool is, in the transition configuration of the atraumatic device, characterized by an outside diameter of no greater than approximately 7 mm at any point along the longitudinal axis of the surgical retractor tool.

The foregoing and other features and advantages of the invention will become apparent from the detailed description below, in light of the accompanying drawings.

### BRIEF DESCRIPTION OF THE DRAWINGS

FIG. 1 is a perspective view of the surgical retractor tool of the present invention according to a first embodiment, and shown in the transition configuration thereof.

FIG. 2 is a perspective view of the surgical retractor tool of FIG. 1, shown in the deployed configuration thereof.

FIG. 3 depicts in perspective the surgical retractor tool of FIG. 1, shown in the deployed configuration and with the connector thereof in the process of being adjusted in length.

FIG. 4 is a detailed perspective view of the surgical retractor tool of FIGS. 1-3, with the elastic portion thereof being depicted in an elastically deformed condition to detail the change in appearance of the visual strain indicator.

FIG. 5 is an exploded perspective view of the retractor tool of FIGS. 1-4, with the elastic portion of the connector shown in separation from the rest of the tool.

FIG. 6 is a detailed view of the retractor tool of FIG. 1, with the first section of the atraumatic device shown partly in phantom to better illustrate the releasable locking joint between the first and second sections of the atraumatic device. In FIG. 6, the first and second sections of the atraumatic device are shown in the transition configuration thereof.

FIG. 7 reproduces the view of FIG. 6, but with the first and second sections of the atraumatic device being shown in the deployed configuration thereof.

FIG. 7B reproduces, in an exploded view, the atraumatic device of FIG. 7.

FIGS. 8A and 8B depict in perspective views an alternative embodiment of an anchor for the retractor tool of the present invention.

FIG. 9A is a perspective view of the surgical retractor tool of the present invention according to a second embodiment, and shown in the transition configuration thereof.

FIG. 9B is a transverse cross-sectional view taken from FIG. 9A.

FIG. 10 is a perspective view of the surgical retractor tool of FIG. 9, shown in the deployed configuration thereof.

FIGS. 11A-11D depict several optional variations of the surgical retractor tool of FIGS. 9A through 10.

FIG. 12 is a perspective view of a surgical retractor tool according to a further embodiment, shown in the transition configuration thereof.

### DETAILED DESCRIPTION

Referring now to the drawings, where like numbers indicate like or corresponding parts throughout the several views, there is shown a surgical retractor tool, such as may be used for endoscopic and minimally-invasive surgeries, including, by way of non-limiting example, laparoscopic and robotic surgeries.

As will be better understood from the following disclosure, including the drawings, the surgical retractor tool of the present invention is advantageously adapted for introduction entirely through a surgical port, for instance a trocar, into the body of a patient during minimally-invasive surgery, whereafter it can be transformed to a configuration suitable for use during surgery as a retractor having an atraumatic retractor device. Heretofore, such a surgical tool has been unknown.

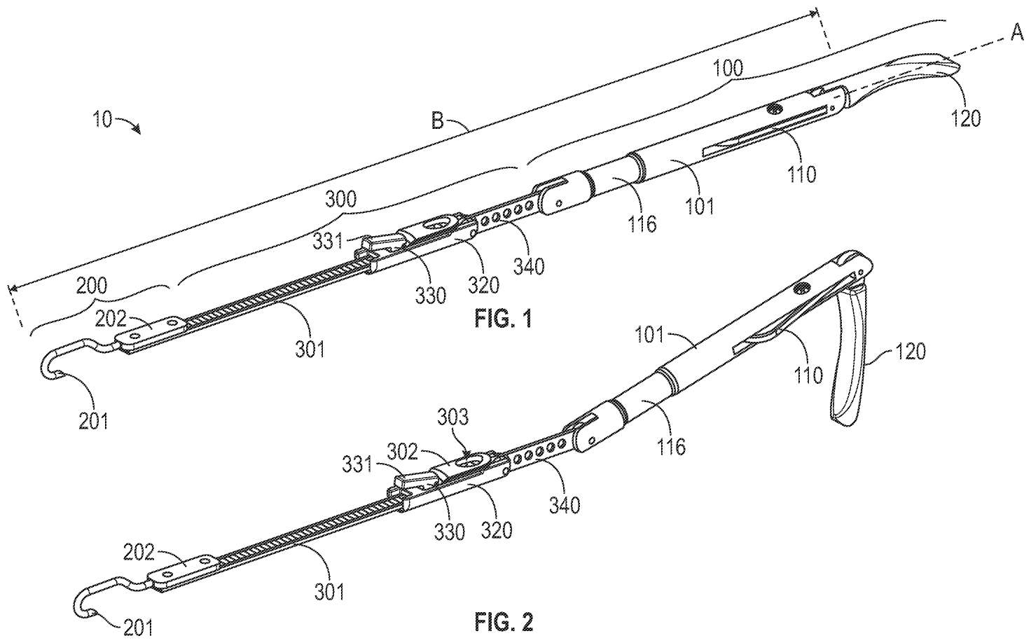

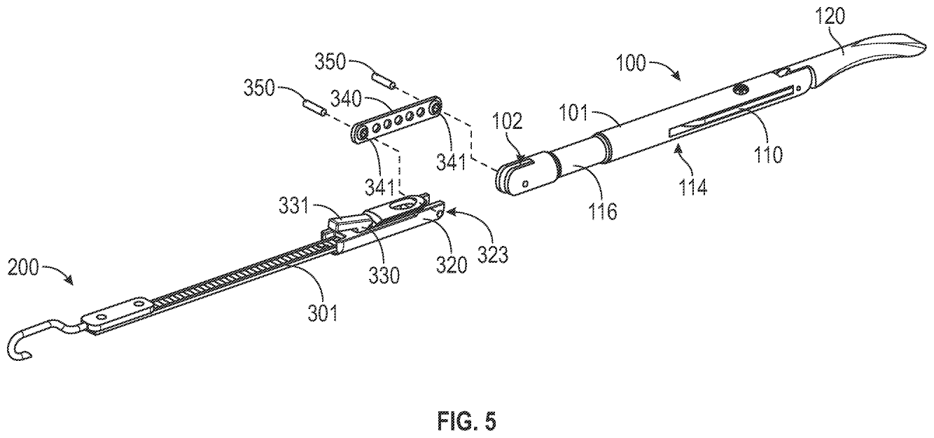

With reference being first had to the embodiment of FIGS. 1-7B as representative of several embodiments of the present invention in more essential respects, the device of the present invention generally comprehends a surgical retractor tool (indicated generally at 10) comprising each of an atraumatic device 100 at a one end of the tool, an anchor 200 at an opposite end of the tool, and a connector 300 coupled at a first end thereof to the atraumatic device 100 and coupled at a second, opposite end thereof to the anchor 200.

The term “atraumatic device” refers to a blunt instrument designed to avoid puncturing or damaging tissue or other anatomical portions of the patient when used as intended.

The atraumatic device 100 is transformable between a transition configuration (shown in FIG. 1), according to which the atraumatic device is characterized by a substantially linear shape defined along a longitudinal axis A of the atraumatic device, and a deployed configuration (shown in FIG. 2), according which the atraumatic device 100 is transformed along the longitudinal axis A to define a non-linear shape that is configured to releasably engage a first anatomical portion of a patient. In the transition configuration of the atraumatic device, the entire surgical retractor tool 10 is characterized by a substantially linear shape defined along a longitudinal axis (indicated by the line B running parallel thereto) of the retractor tool extending from an extreme end of the anchor 200 to an extreme end of the atraumatic device 100.

The anchor 200 is configured to be releasably secured to a second anatomical portion of the patient. Depending on the particular surgical application, embodiments of the anchor can comprise any of a bulldog clamp, a claw, a pointed hook, or other anchor types commonly employed in surgical applications.

According to a first embodiment, shown in FIGS. 1-3 and 5, anchor 200 takes the form of a hook with an end 201 adapted to be releasably secured by traumatic engagement to an anatomical portion of the patient. A base portion 202 is secured to an end of the connector 300, as hereafter described. Anchor 200 of this embodiment may be made of stainless steel or other material suited to the surgical application of the present invention.

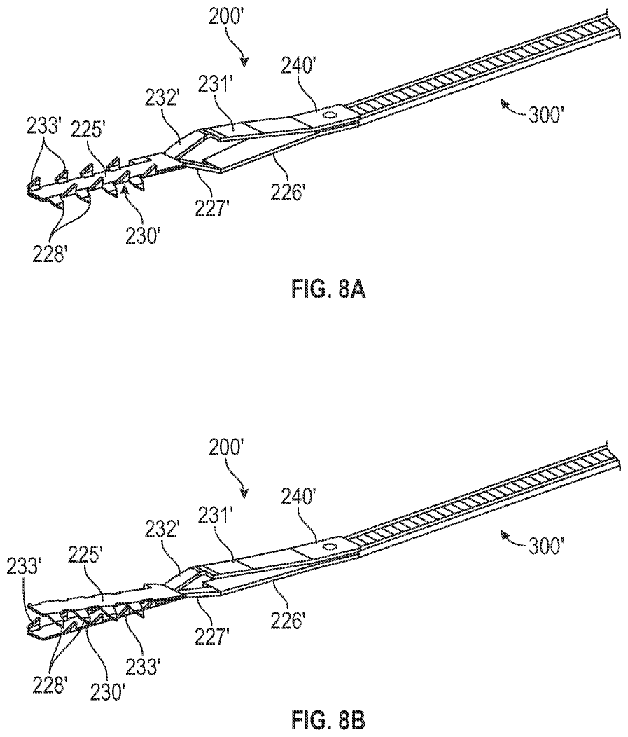

Per another embodiment, shown in FIGS. 8A and 8B, anchor 200′ is configured as a clamp that is manually actuatable between a closed, clamped position (shown in FIG. 8A) and an open, unclamped position (FIG. 8B). Still more particularly, the clamp of anchor 200′ is depicted as a bulldog clamp of conventional construction, being comprised of first 225′ and second 230′ jaws biased to the closed, clamped position of the anchor. Anchor 200′ is attached at an end 240′ opposite the jaws to connector 300′. Each jaw 225′, 230′ is disposed at the end of an actuation region comprised of, respectively, outwardly angled regions 226′, 231′ which transition to inwardly angled regions 227′, 232′ that terminate at jaws 225′, 230′. Each jaw and its associated actuation region is formed from a single piece of material, such as stainless steel, for instance. Alternatively, the entire clamp may be formed from a single piece of material, being bent or folded, for instance, at the point of attachment to the connector 300′. In either case, it will be appreciated that the aforedescribed configuration of the clamp results in movement of each jaw oppositely away from the other when each actuation region is manually urged toward the other. It will also be appreciated that, by the foregoing configuration, the actuation regions and, correspondingly, their associated jaws, are biased toward the closed configuration of the clamp.

With continuing reference to FIG. 8, each jaw 225′, 230′ includes a plurality of teeth 228′, 233′, respectively, arranged to traumatically engage the second anatomical portion of the patient when the clamp is closed over such anatomical portion. In the closed position (FIG. 8), the teeth 228′, 233′, of each jaw 225′, 230′ will be seen to project oppositely away from each other.

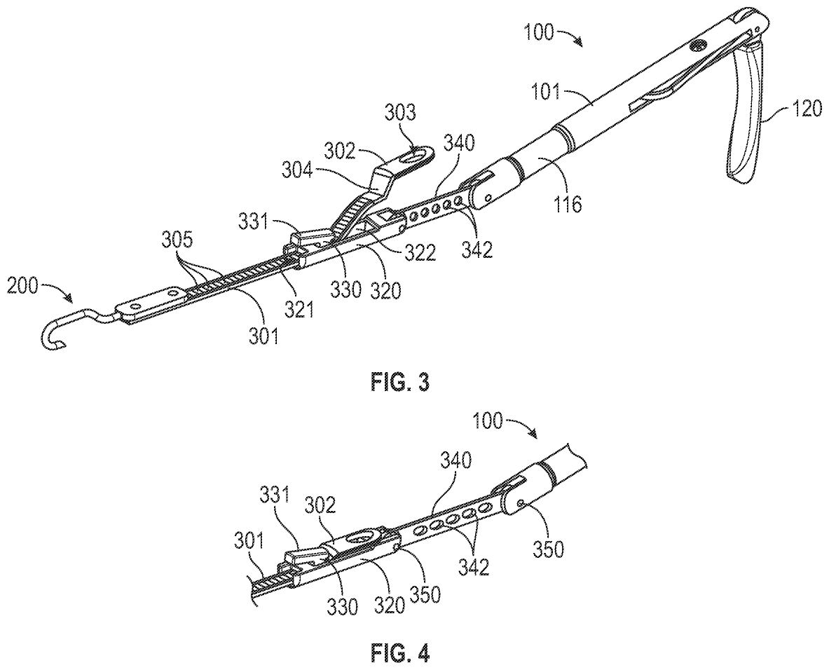

Referring again to FIGS. 1-5, connector 300 has a length that is manually adjustable to adjust an overall length of the surgical retractor tool 10. Per the illustrated embodiment more particularly, connector 300 comprises a first, adjustable strap portion 301. Strap portion 301 is fixedly connected at one end to the anchor 200 and terminates at an opposite, free end disposed nearer the atraumatic device 100. In the illustrated embodiment, free end includes a tab 302 having an opening 303 defined therethrough, the opening 303 being dimensioned to receive part of a surgical tool, such as graspers, scissors, etc., therein to facilitate adjustment of the connector 300 in the manner hereafter described. As best shown in FIG. 3, strap portion 301 includes an upwardly angled portion 304 which defines a transition between the majority of strap portion 301 and the tab 302. Tab 302 extends outwardly away from angled portion 304 and toward the atraumatic device 100 in a plane generally parallel to the plane of the majority of the strap portion 301.

An upper surface of the majority of the strap portion 301—the area between the anchor 200 and angled portion 304—is provided with a plurality of spaced-apart teeth 305 arranged seriatim along the strap portion 301. Per convention according to the operation of the ratchet mechanism hereafter described, each such tooth is defined by an angled face and a vertical face, with the angled face of each tooth preceding the vertical face in the direction of shortening travel of the connector's strap portion 301 which, as explained further below, is toward the atraumatic device 100.

Connector strap portion 301 is, per the illustrated embodiment, formed of nylon.

Connector 300 further comprises a somewhat cylindrically-shaped housing 320 into a first opening 321 of which is received the strap portion 301 with, as shown in FIGS. 1-3, at least the tab 302 of free end emerging from a second opening 322. Housing 320 supports a pawl 330 including one or more teeth (not visible). Pawl 330 is biased into engagement between the teeth thereof and the teeth 305 of the connector strap portion 301. Per convention, the one or more teeth of pawl 330 will be understood to each have a shape that is complimentary to the shape of teeth 305 of strap portion 301, such that engagement of the one or more teeth of pawl 330 with teeth 305 of strap portion 301 will result in the corresponding vertical faces thereof abutting to resist movement of the connector strap portion 301 in the direction of lengthening travel which, as explained further below, is away from the atraumatic device 100.

Conversely, the angled face of each tooth 305 of the strap portion 301 is adapted to permit each tooth 305 to pass freely over the corresponding angled face of each tooth of the pawl 330 in the direction of shortening travel of the strap portion 301. More specifically, it will be understood that shortening travel of the connector strap portion causes the angled surface of each tooth thereof to urge pawl 330 outwardly away from the strap portion 301 until pawl passes over the angled surface, whereupon the bias acting on pawl 330 urges the one or more teeth thereof back into engagement with corresponding teeth of the connector strap portion 301.

By the foregoing, it will be appreciated that adjustment of the connector 300 relative to the atraumatic portion 100 is accomplished by engaging the free end of connector strap portion 301, such as with graspers positioned at least partially within the opening 303 of tab 302, and manually pulling the free end toward the atraumatic device, thereby advancing the teeth of strap portion 301 past the pawl 330 until a desired overall length of the retractor tool 10 is realized. When the desired adjustment in length of the strap portion 301 is achieved, the user simply stops manually pulling the free end of strap portion. Thereafter, engagement between the one or more teeth of pawl 330 and teeth of the strap portion 301 acts in the manner heretofore described to resist lengthening movement of the strap portion.

Pawl 330 may be biased by any suitable means, including by a spring or a living hinge formed as part of the pawl 330 (such as commonly employed in conventional plastic cable-ties).

To effect lengthening of the connector 300, pawl 330 is provided with a release lever 331 which, when manually depressed toward the housing 320 (such as by means of graspers or other surgical tool), urges the one or more teeth of pawl 330 out of engagement with the teeth of connector strap portion 301. Release lever 331 acts against the bias of pawl 330, such that the connector strap portion 301 can be manually pulled in a direction away from the atraumatic device 100—i.e., the lengthening direction of connector 300—so long as release lever 331 remains depressed.

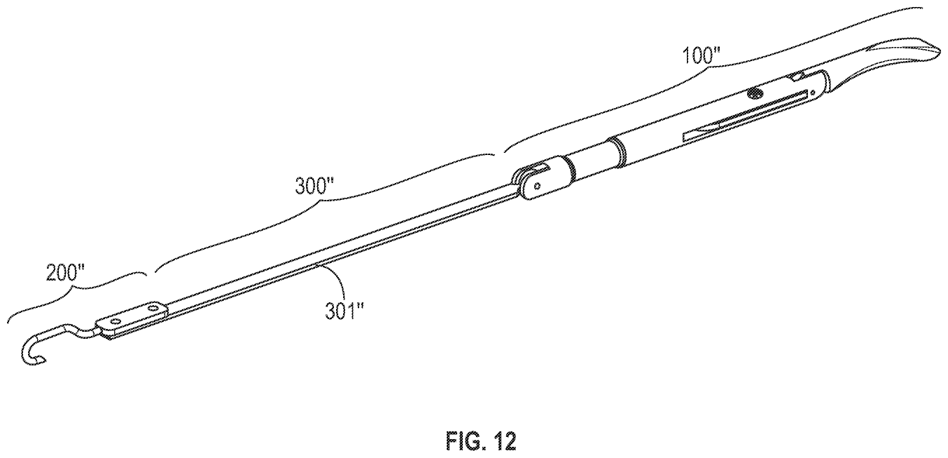

Of course, it will be understood by those skilled in the art that the mechanism for adjusting the overall length of the surgical tool via the connector may be other than as described above. Any of a variety of other mechanism could be substituted for that of the illustrated embodiment to equivalent effect. Alternatively, it is contemplated that the retractor tool of the present invention may also comprise a connector which is of fixed length; i.e., not adjustable in length via any mechanism, including as described above in respect of the embodiment of FIGS. 1-7B. Such an alternative embodiment of the present invention is depicted in FIG. 12. The connector 300″ according to such an alternative embodiment may, by way of non-limiting example, comprise a strap 301″ of nylon secured at either end to each of the anchor 200″ and atraumatic device 100″. The manner of such securement could be any conventional means, including as described herein.

Referring to FIGS. 1-5, connector 300 according to one illustrated embodiment also comprises an elastic portion 340 that is temporarily deformable in at least the longitudinal axis B of the surgical retractor tool. Elastic portion 340 is interposed between the housing 320 and atraumatic device 100, being pivotally connected to each by hinge joints. Each such hinge joint comprises an eye portion 341 disposed at each of opposite ends of the elastic portion 340. Each eye portion 341 is received in a corresponding fork portion 323, 102 of one of the housing 320 or atraumatic portion 100, respectively. Transversely oriented openings through each of the eye and fork portions are coaxially aligned when each eye portion 341 is seated in a corresponding fork portion. A pin 350 disposed through each of these aligned openings secures the fork and eye portions together for pivotal movement about the pin.

Elastic portion 340 includes, in the illustrated embodiment, a strain indicator configured to identify stretching of the elastic portion due to forces acting on one or both of the anchor 300 and/or the atraumatic portion 100. As depicted, the strain indicator takes the form of a series of openings 342 which will visibly distort (shown in FIG. 4) as the elastic portion 340 is temporarily deformed in response to forces acting to stretch the retractor tool from one or both ends thereof.

According to the illustrated embodiment, elastic portion 340 is formed of a thermoplastic elastomer. The elasticity of this portion 340 permits temporary deformation by lengthening. In use, this aspect of the present invention will be understood to permit an end (whether the anchor or the hook of the traumatic portion) to be secured to an anatomical portion of a patient, whereafter the retractor tool can be stretched to permit the opposite end to be secured to another anatomical portion of the patient. As the material of the elastic portion 340 is urged back to its default, unbiased condition, it will be appreciated that at least one end of the retractor—whether the anchor or the hook of the atraumatic device—will be urged in the same direction, thus enhancing the degree of retraction of the associated anatomical portion of the patient.

While, per the illustrated embodiment, connector 300 is shown to comprise elastic portion 340, it will be appreciated that the retractor tool 10 of this invention need not include this feature. Instead, atraumatic portion 100 may be hingedly connected directly to the housing 320 (substituting, for instance, an eye portion for either of the fork portions 102, 323 heretofore described). Alternatively, it should be understood that a hinged connection between the atraumatic device 100 and connector 300 could be forgone entirely. Instead, connector housing 320 and atraumatic device 100 could be combined into a unitary structure.

Still further, it is contemplated that, in an alternative embodiment, the retractor tool of the present invention may incorporate a connector which is entirely, or substantially, the elastic portion described above. The connector according to such an alternative embodiment could be secured at either end to each of the anchor and atraumatic device by any conventional means, including as described herein.

With reference now being had to FIGS. 1-7B, the construction and operation of the atraumatic device 100 according to a first embodiment will be better understood.

Per this first embodiment, atraumatic device 100 comprises first 101 and second 120 sections coupled by a hinge joint at which the atraumatic device is transformable between the transition configuration and the deployed configuration.

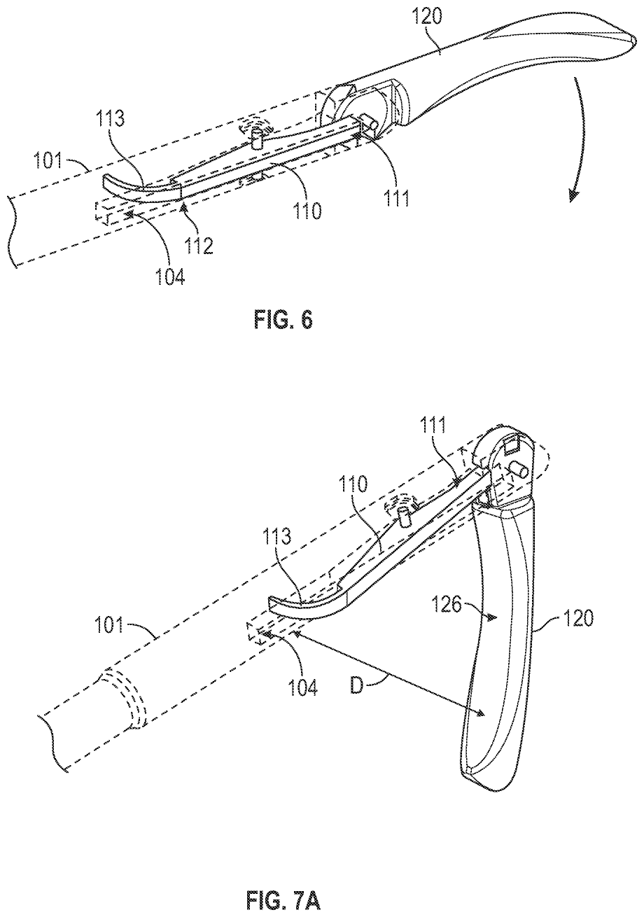

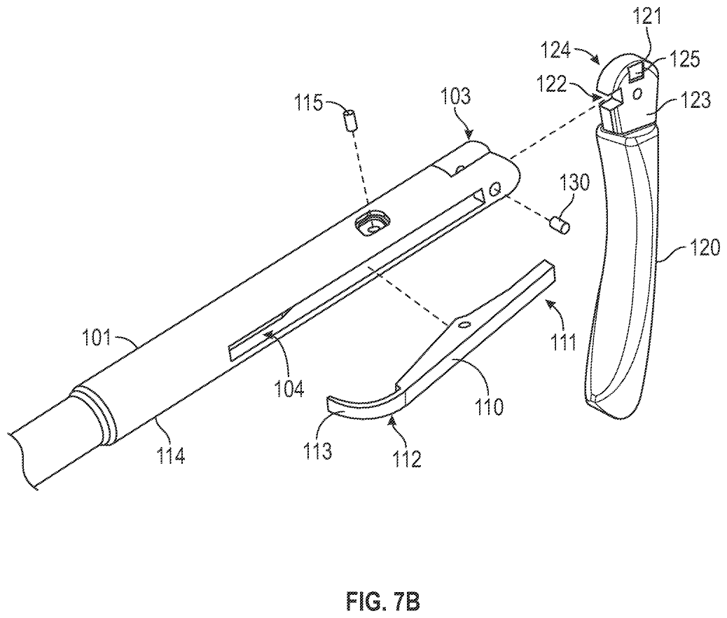

FIGS. 6-7B depict hinge joint in detail. As shown, an end of first section 101 defines a fork end 103 of the joint, while the adjacent end of second section 120 defines an eye end 121 of the joint. The eye end is received in the fork end. Openings through each of the eye and fork ends are coaxially aligned when the eye end is seated in the fork end. A pin 130 disposed through these openings secures the fork and eye ends together for pivotal movement about the pin.

The eye end 121 includes a cut-out area 122 which opens radially outwardly. As depicted, cut-out area 122 is defined entirely between opposing lateral surfaces 123, 124 of the eye end. A recess 125 is also defined in a surface of the eye end, the recess 125 also opening radially outwardly from the eye end as shown. Recess 125 is characterized by a shallow depth in the lateral surface 123 of eye end. With the pivot pin 130 defining the center-point, the angular distance between cut-out area 122 and recess 125 corresponds to the degree of travel of the second section 120 between the transition and deployed configurations of the atraumatic device.

First section 101 includes a locking lever 110 disposed in a recessed opening 104 therein, the locking lever 110 being pivotally connected to first section 101 by a pivot pin 115. Locking lever 110 includes a head end 111 and a tail end 112. Head end 111 is dimensioned to be fully received in cut-out area 122 and partially received in recess 125, depending on the angular orientation of the second section 120 relative to the first section 101. More particularly, head end 111 is fully received in cut-out area 122 when atraumatic device 100 is in the deployed configuration; head end 111 is partially received in recess 125 when atraumatic device 100 is in the transition configuration.

Tail end 112 includes a spring 113 which engages a surface of the recessed opening 104 to bias locking lever 110 in the position of FIG. 7; that is, a position of engagement between head end 111 and cut-out area 122. Per the illustrated embodiment, spring 113 is part of the locking lever 110, being configured to impart a spring bias by its' shape.

The degree of pivotal motion of locking lever 110 relative to first section 101 is such that, when locking lever 110 is, by manual depressing the section of locking lever 110 proximate tail end 112, head end 111 moves away from first section 101 and out of engagement with the cut-out area 122.

As will be appreciated from the drawings, when head end 111 is fully received in cut-out 122, opposing surfaces of each of head end 111 and cut-out 122 will abut to prevent more than minimal, if any, rotation of second section 120 in either a clockwise or counter-clockwise direction about pivot point 130.

Per the illustrated embodiment, the depth of recess 125 is such that, when head end 111 is received therein, second section 120 will not freely rotate from the transition configuration of atraumatic device 100. This is shown in FIG. 6, with respect to which it can be seen that, in this configuration, locking lever 110 is oriented within the recessed opening 104 so as to be substantially flush with the outer surface of first section 101. However, the depth of recess 125 is preferably—though not necessarily—such that the application of manual force, including via surgical tools such as graspers, to rotate second section 120 relative to first section 101 will urge head end 111 outwardly against the biasing force of spring 113 and out of engagement with recess 125. In this manner, rotational movement of second section 120 to change atraumatic device from the transition configuration to the deployed configuration can be accomplished without having to actuate locking lever 110.

It will be appreciated that the construction described above defines two predefined angular relationships between the first 101 and second 120 sections of the atraumatic device 100: A first angular relationship (FIG. 6) corresponding to the transition configuration, in which head end 111 of locking lever 110 is received in recess 125; and a second angular relationship (FIG. 7A) corresponding to the deployed configuration, in which the head end 111 of locking lever 110 is received in cut-out 122. It will also be appreciated that the construction described above provides a positive indication that the adjacent first and second sections 101, 120 have reached their predefined angular relationships, whether in the transition or deployed configurations, thereby facilitating easy transformation of the atraumatic device between these configurations.

Those skilled in the art will understand, with the benefit of this disclosure, that one or more additional cut-out and/or recessed areas may be defined in the eye end 121 in order to provide more than two predefined angular relationships between the first 101 and second 120 sections of the atraumatic device 100.

As shown, second section 120 is characterized by a curved shape along its longitudinal axis. This curved shape defines an inwardly curved surface (indicated generally at 126) which, in the deployed configuration, imparts a hook-shaped appearance to the atraumatic device.

The internal dimension D of this hook-shape defined by the angular relationship of the first 101 and second 120 sections in the deployed configuration will depend on the length and geometry of these sections, as well as the predefined angular relationship of the deployed configuration, as those skilled in the art will understand. In the embodiments shown in FIGS. 1-7B, it is contemplated, for instance, that internal dimensions D of 10 mm, 20 mm, or 32 mm may be provided in various forms of the present invention. It will of course be understood that other internal dimensions can also be engineered, according to the needs of the user with respect to the retractor tool 10.

With continuing reference to FIGS. 1-2 and 6-7, it will be seen that the atraumatic device 100 is characterized by smooth surfaces for releasably engaging the first interior anatomical portion of the patient. More specifically, these surface in the illustrated embodiment include at least the inwardly curved surface 126 of second section 120, and at least the surface 114 of first section 101. These smooth surfaces 126, 114 will be understood to at least minimize the potential for the device to catch on, or otherwise traumatize, the anatomical portion of the patient with which it is in contact.

With reference now being had to FIGS. 1-5, it can be seen that the atraumatic device 100 first section 101 includes at least one control area 116 to facilitate manipulation of the retractor tool, whether by hand or via a surgical tool such as graspers, etc. Optionally, this control area can be knurled or otherwise textured.

The atraumatic device and its various components as described herein may be made at least substantially of polycarbonate. However, it will be appreciated that other materials likewise suited to the surgical environment of the present invention may be substituted without departing from the broader aspects of the invention.

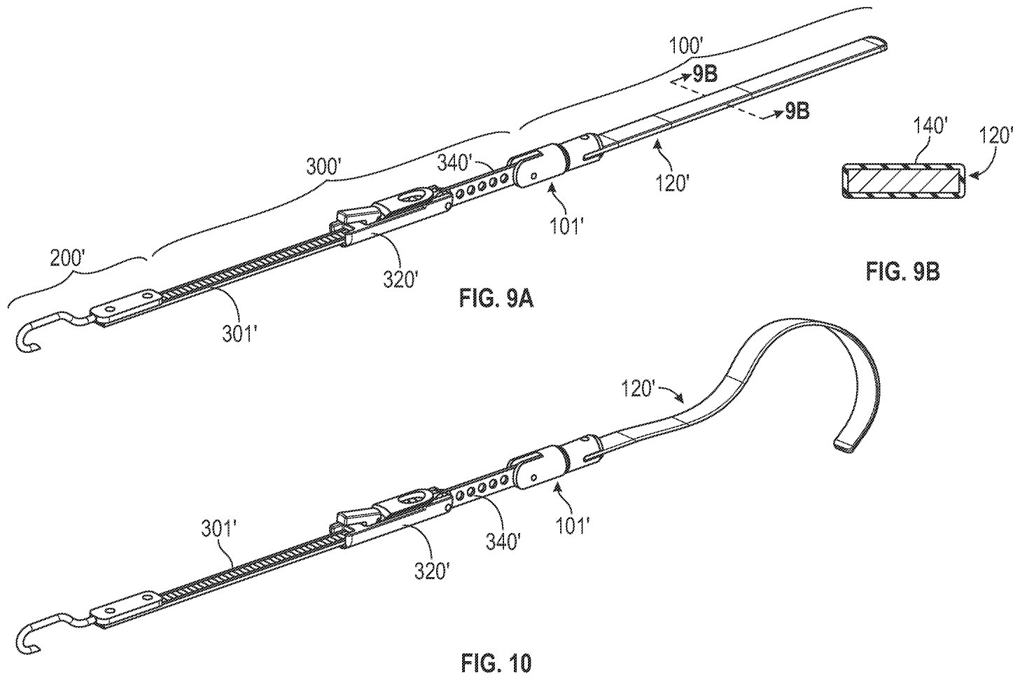

Turning now to FIGS. 9A-10, there is shown an embodiment of the present invention according to which the atraumatic device 100′ comprises a first section 101′ and a second section 120′ fixedly connected thereto, the second section 120′ taking the form of a generally flat, thin length of monolithic material which is manually deformable along its length to transform the atraumatic device between the transition configuration (FIG. 9A) and the deployed configuration (exemplified in FIG. 10). Per the illustrated embodiment, the material of second section 120′ is stainless steel. Preferably, though not necessarily, the deformable material of second section 120′ is overcoated 140′ with an elastomeric material such as, by way of example only, silicone.

First section 101′ is secured to connector 300—such as to the elastic portion 340′ as shown in FIGS. 9A-10—by a hinge joint in the manner heretofore described.

Second section 120′ is configured to be manually deformable via surgical tools such as, for example, graspers. For instance, a first set of graspers can be utilized to hold the second section 120′ in place while a second set of graspers is utilized to effect a desired bend in the deformable material of the second section.

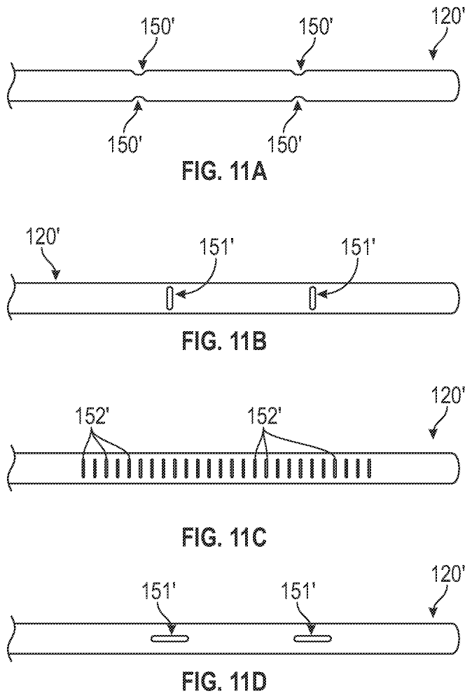

To further facilitate manual deformation of the device 100′, the deformable material of second section 120′ may optionally be provided with one or more physical features, such as lateral cut outs 150′ (FIG. 11A) and/or interior cut-outs 151′ (FIGS. 11B, 11D) to weaken specific areas of the deformable material, areas of indentation 152′ (FIG. 11C), etc. To the extent that the second section 120′ is overcoated, these physical features, when not otherwise visible, could be identified by printing indicia on the overcoating.

As with the embodiment of the atraumatic device first described above, the atraumatic device 100′ of this embodiment is, as shown, characterized by smooth surfaces for releasably engaging the first interior anatomical portion of the patient. As before, these smooth surfaces will be understood to at least minimize the potential for the device to catch on, or otherwise traumatize, the anatomical portion of the patient with which it is in contact. Similarly, the silicone overcoating 140′ will be understood to minimize damage to the patient from sharp or abrupt edges of the underlying metallic material, including as created during bending thereof in use.

Other than as described above, it will be appreciated from the accompanying drawings that the retractor tool of the embodiment of FIGS. 9-10 is otherwise the same as the retractor tool described in relation to FIGS. 1-8.

The surgical retractor tool 10 of the present invention can be dimensioned to be introduced, in the transition configuration thereof, through a laparoscopic port into the patient's body (e.g., abdomen or thoracic cavity) during minimally-invasive surgery to allow internal retraction of organs, vessels or other tissue in an atraumatic fashion. More particularly, the surgical retractor tool is dimensioned so that, in the transition configuration of the atraumatic device, the surgical retractor tool can be received into the patient's body through a lumen of a surgical port, such as, for instance, a trocar. Once in the body, the tool 10 can be transformed to the deployed configuration in the manner heretofore described for the several embodiments, it being understood that the deployed configuration is characterized by dimensions larger than the size of the port in the patient. In one non-limiting example, the surgical retractor tool is characterized by an outside diameter of no greater than approximately 7 mm at any point along the longitudinal axis of the surgical retractor tool, such outside diameter being smaller than the inside diameter of the lumen of a conventional surgical port used for minimally-invasive surgery.

Of course, it is also contemplated that the retractor tool of the present invention may also be only partly deployed in the body of the patient. For instance, the atraumatic device could also be anchored externally (extracorporeally), exiting a surgical port and secured outside the patient's body or with a percutaneous suture manipulated externally for adjustment of tension/retraction.

Various terms are used to refer to particular system components. Different entities may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

The above discussion is meant to be illustrative of the principles and various embodiments. Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims. Numerous variations and modifications will become apparent to those skilled in the art once the above disclosure is fully appreciated. It is intended that the following claims be interpreted to embrace all such variations and modifications.

1. A surgical retractor tool, comprising:
an atraumatic device transformable between a transition configuration, in which the atraumatic device is characterized by a substantially linear shape defined along a longitudinal axis of the atraumatic device, and a deployed configuration, in which the atraumatic device is transformed along the longitudinal axis of the atraumatic device to define a non-linear shape configured to releasably engage a first anatomical portion of a patient;
an anchor configured to be releasably secured to a second anatomical portion of the patient; and
a connector coupled at a first end to the atraumatic device and coupled at a second, opposite end to the anchor;
wherein, in the transition configuration of the atraumatic device, the surgical retractor tool is characterized by a substantially linear shape defined along a longitudinal axis of the surgical retractor tool extending from an end of the anchor to an end of the atraumatic device.

2. The surgical retractor tool of claim 1, wherein the connector comprises a length that is manually adjustable relative to the atraumatic device to adjust an overall length of the surgical retractor tool.

3. The surgical retractor tool of claim 1, wherein the atraumatic device is hingedly coupled to the connector.

4. The surgical retractor tool of claim 1, wherein the connector further comprises an elastic portion that is temporarily deformable in at least the longitudinal axis of the surgical retractor tool.

5. The surgical retractor tool of claim 4, wherein the elastic portion of the connector includes a strain indicator configured to identify temporary deformation of the elastic portion.

6. The surgical retractor tool of claim 4, wherein the atraumatic device is coupled to the elastic portion.

7. The surgical retractor tool of claim 1, wherein, in the deployed configuration, the non-linear shape of the atraumatic device defines a hook configured to releasably engage the first anatomical portion of the patient.

8. The surgical retractor tool of claim 7, wherein the atraumatic device comprises at least one hinge joint at which the atraumatic device is transformable between the transition configuration and the deployed configuration.

9. The surgical retractor tool of claim 8, wherein the at least one hinge joint is provided between first and second sections of the atraumatic device.

10. The surgical retractor tool of claim 9, wherein the at least one hinge joint comprises a releasable locking joint configured to selectively lock the at least one hinge joint in each of the transition and deployed configurations of the atraumatic device.

11. The surgical retractor tool of claim 1, wherein the atraumatic device is characterized by smooth surfaces for releasably engaging the first anatomical portion of the patient.

12. The surgical retractor tool of claim 1, wherein the anchor is configured to be traumatically secured to the second anatomical portion of the patient.

13. The surgical retractor tool of claim 12, wherein the anchor is a clamp that is manually actuatable between a closed, clamped position and an open, unclamped position.

14. The surgical retractor tool of claim 1, wherein, in the transition configuration of the atraumatic device, the surgical retractor tool is dimensioned to be received into a patient through a surgical port.

15. The surgical retractor tool of claim 14, wherein, in the transition configuration of the atraumatic device, the surgical retractor tool is characterized by an outside diameter of no greater than approximately 7 mm at any point along the longitudinal axis of the surgical retractor tool.

16. The surgical retractor tool of claim 1, wherein the atraumatic device comprises a monolithic length of material which is manually deformable to transform the atraumatic device between the transition configuration and the deployed configuration.

17. The surgical retractor tool of claim 16, wherein the monolithic length of material is overcoated with an elastomeric material.

18. The surgical retractor tool of claim 17, wherein the elastomeric material is silicone.

19. The surgical retractor tool of claim 16, wherein the connector comprises a length that is manually adjustable relative to the atraumatic device to adjust an overall length of the surgical retractor tool.

20. The surgical retractor tool of claim 16, wherein the atraumatic device is hingedly coupled to the connector.

21. The surgical retractor tool of claim 16, wherein the connector further comprises an elastic portion that is temporarily deformable in at least the longitudinal axis of the surgical retractor tool.

22. The surgical retractor tool of claim 21, wherein the elastic portion of the connector includes a strain indicator configured to identify temporary deformation of the elastic portion.

23. The surgical retractor tool of claim 21, wherein the atraumatic device is coupled to the elastic portion.

24. The surgical retractor tool of claim 16, wherein the atraumatic device is characterized by smooth surfaces for releasably engaging the first anatomical portion of the patient.

25. The surgical retractor tool of claim 16, wherein the anchor is configured to be traumatically secured to the second anatomical portion of the patient.

26. The surgical retractor tool of claim 25, wherein the anchor is a clamp that is manually actuatable between a closed, clamped position and an open, unclamped position.

27. The surgical retractor tool of claim 16, wherein, in the transition configuration of the atraumatic device, the surgical retractor tool is dimensioned to be received into a patient through a surgical port.

28. The surgical retractor tool of claim 27, wherein, in the transition configuration of the atraumatic device, the surgical retractor tool is characterized by an outside diameter of no greater than approximately 7 mm at any point along the longitudinal axis of the surgical retractor tool.
